(19) United States
(12) Patent Application Publication
(10) Pub. No.: US 2011/0106170 A1
(43) Pub. Date: May 5, 2011

(54) TACK FOR SPINE FIXATION

(76) Inventor: Timothy E. DOERR, Boise, ID (US)

(21) Appl. No.: 12/915,014

(22) Filed: Oct. 29, 2010

Related U.S. Application Data
(63) Continuation-in-part of application No. 12/541,912, filed on Aug. 14, 2009.
(60) Provisional application No. 61/088,793, filed on Aug. 14, 2008.
(60) Provisional application No. 61/097,095, filed on Sep. 15, 2008.
(60) Provisional application No. 61/161,074, filed on Mar. 18, 2009.

(51) Int. Cl. A61B 17/88; A61B 17/86

(52) U.S. Cl. 606/279; 606/329

(57) ABSTRACT

A tack for insertion into facets joints of the human spine includes one or more bioactive materials. The tack is preferably pushed/impacted axially into a hole in the facets, rather than rotated or screwed into the hole/facets. For example, the bioactive material may be outer sidewall(s) made of porous material that receives and/or encourages bone growth into its pores. Or, for example, the bioactive material may be osteobiologic material, demineralized bone matrix (DBM), sponge holding bone morphogenic protein (BMP), allograft bone, or other bioactive material inside an interior space of the tack. Apertures may be provided in the outer wall of a hollow tack to allow bone growth into the interior space of the tack. The tack may have a longitudinal passage, so that the tack may be installed on and slid along a guide-wire in percutaneous surgery that is guided by intraoperative imaging navigation. Preferably, the tack is not threaded, and is installed with little, and preferably no, rotation of the tack on its longitudinal axis.

This application is a continuation-in-part of Non-Provisional application Ser. No. 12/541,912, filed Aug. 14, 2009, which claims benefit of Provisional Application Ser. No. 61/088,793, filed Aug. 14, 2008, Provisional Application Ser. No. 61/097,095, filed Sep. 15, 2008, and Provisional Application Ser. No. 61/161,074, filed Mar. 18, 2009, the entire disclosures of which provisional and non-provisional applications are incorporated herein by this reference.

### BACKGROUND OF THE INVENTION

1. Field of the Invention

The invention relates generally to apparatus for fixation of portions of the human spine, and, more particularly, to apparatus for fixing one vertebra to another, or a vertebra to the sacrum. The preferred apparatus is a tack, made from or comprising bioactive materials, which is axially inserted into holes in facets of said vertebra(e) and/or sacrum, rather than being screwed into said holes. Said preferred tack is surprisingly the only structure needed for posterior fixation of the spine, and the tack preferably does not connect to, and preferably is not an anchor or fastener for, any supplemental fixation or support structure such as bars, brackets or plates. The preferred tack is used on posterior surfaces of the vertebra(e) and sacrum, preferably in combination with an anterior fixation device such as a fixation plate that is attached to anterior surfaces of said vertebra(e) and/or sacrum.

2. Related Art

Screws and/or plates and arms have been used on the spine to fix portions of the spine together and/or to perform other repair. For example, see Vichard (U.S. Pat. No. 5,318,567); Puno, et al. (U.S. Pat. No. 5,360,431); Ray (U.S. Pat. No. 5,527,312); Cornwall, et al. (U.S. Pat. No. 6,485,518); Berry, et al. (U.S. Patent Application 2006/0276788 A1); Culbert, et al. (U.S. Patent Application 2007/0118132 A1); and Berg, et al. (2008/0015585). These systems tend to be complex and include screwing of threaded members into bone.

The inventor believes that there is a need for an improved implant/apparatus for spine fixation that is simple in structure and minimally-invasive. The inventor believes that there is a need for an improved implant/apparatus that comprises bioactive materials that allow or encourage bone growth into the implant/apparatus, preferably resulting in bone growth all the way through the implant/apparatus and/or replacement of the material of the implant/apparatus by bone growth. The inventor believes that there is a need for an improved implant/apparatus that is minimally invasive but that is sufficiently strong and durable so that it may be forced into holes in the vertebra(e) and/or sacrum without breaking.

### SUMMARY OF INVENTION

The invention comprises a tack for insertion into facets of the human spine, wherein the tack comprises one or more bioactive materials. The tack is preferably pushed/impacted/tapped axially into holes in said facets, rather than rotated or screwed into said holes/facets. In preferred embodiments, the tack is installed at the posterior side of the lumbar region of the spine, to either fix facets of two vertebrae together or to fix the facets of the lowermost vertebra to facets of the sacrum.

Preferably, the tack is not threaded, and is installed with little, and preferably no, rotation of the tack on its longitudinal axis. In a first group of embodiments, the tack is made with barbs or other protrusions that resist or prevent the tack from backing out of the holes of the facets. In a second group of embodiments, the tack is made without barbs and without other protrusions, and the material of the tack and its interaction with the bone is sufficient to resist or prevent the tack from backing out of the holes of the facets. In some embodiments, the tack is made substantially of entirely of bioactive materials, for example, a solid (non-hollow) piece of bioactive material. In other embodiments, the tack is made of an outer housing or outer layer and a core wherein one or the other comprises bioactive materials.

It is important and surprising that the preferred embodiments of posterior spine fixation apparatus consist essentially of, and preferably consist only of, one or more tacks inserted into facets of the facet joints, to extend across one or more facet joints of the spine to fix said facet joints. In other words, preferably only said tacks are used to make the facets of the selected facet joint(s) substantially or entirely immovable relative to each other, so that said the vertebra(e)/sacrum of the spine no longer bend/move relative to each other at said selected facet joint(s). The preferred embodiments of posterior spine fixation, therefore, do not include any additional structure implanted into the body, for example, no bars, no plates, no screws, or other structure extending between portions of the vertebra(e) and/or sacrum, or from vertebra to vertebra, or from vertebra to sacrum. The preferred apparatus is surprising effective and its simplicity results in extremely non-invasive apparatus and surgery methods.

### BRIEF DESCRIPTION OF THE DRAWINGS

It may be noted that, in reference to the figures and the tacks shown therein, the terms “top end” and “bottom end” are used for convenience, with the bottom end being the end that leads during insertion into the body, and the top end being opposite the bottom end. It will be understood that this terminology is not necessarily consistent with the orientation of the tacks when in use in the human body.

FIG. 1 is a front (anterior) view of one embodiment of a fixation plate installed on the lowermost vertebra and the sacrum of a lumbar region of a human spine.

FIG. 2A is a rear (posterior) view of the lumbar region of FIG. 1, wherein one embodiment of the invented tack is shown inserted into the inferior facets of the lowermost vertebra and into the superior facets of the sacrum, to fix the lowermost-vertebra-sacrum facet joint.

FIG. 2B is a posterior view of two vertebrae, wherein two tacks according to embodiments of the invention are installed in (“across”) facet joints of said two vertebrae.

FIG. 2C is a left side view of the lower spine, showing use of tacks according to the preferred embodiments between the sacrum and the lowermost vertebra and also at three other locations between vertebrae. Also, anterior plates are shown in two locations, in dashed lines,

FIG. 2D is a left, partial view of a facet joint fixed by an embodiment of the invented tack.

FIG. 3 is a side view of one embodiment of the invented tack installed into a facet joint, specifically into an inferior facet and a superior facet, wherein the bone facets are shown in cross-section. Bone-gripping protrusions extend from the tack axial side surface 360 degrees around the tack.

FIG. 4 is a side view of another embodiment of the invented tack, wherein the protrusions are intended to abut/grip the wall surface of a hole drilled across the facet joint when the tack is inserted axially preferably without rotation of the tack.

FIG. 5A is a side view of another embodiment of the invented tack, which has protrusions extending from left and right sides of the tack, but not extending from locations 360 degrees around the tack.

FIG. 5B is a side view of the embodiment of the invented tack shown in FIG. 3, prior to being installed across the facet joint.

FIG. 6 is a side view of another embodiment of the invented tack, wherein the (bottom) tip of the tack is rounded, protrusions extend from the side surface of the tack near the tip, and axial slots are provided near the top end of the tack.

FIG. 7 is a side view of another embodiment of the invented tack, wherein multiple, shelf-like protrusions extend from the tack axial side surface in four locations around the tack (for example, spaced generally 90 degrees).

FIGS. 8 and 10 are side views of another embodiment of the invented tack, wherein FIG. 8 shows the tack installed into a facet joint, specifically into an inferior facet and a superior facet, wherein the bone facets are shown in cross-section.

FIG. 10 shows the tack prior to installation. This tack is similar to that shown in FIGS. 3 and 5B, but comprises an enlarged top end.

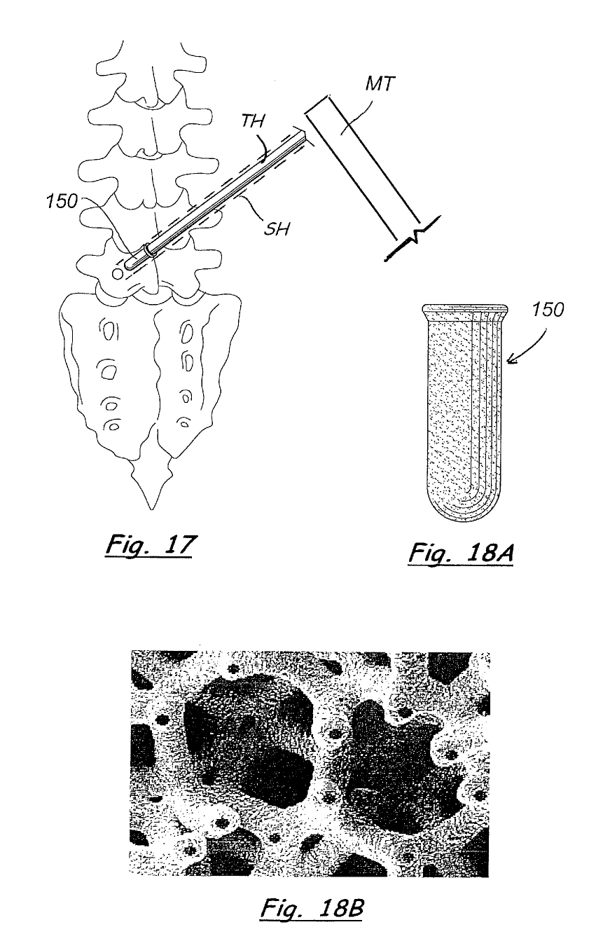

FIGS. 9, 11 and 12 are side views of alternative embodiments of the invention, similar to the tacks in FIGS. 4, 6, and 7, respectively, but with enlarged upper ends for cooperation with a tack holder or impact tool.

FIG. 13 is a side view of another embodiment of the invented tack installed into a facet joint, wherein the bone facets are shown in cross-section. This tack comprises no barbs or protrusions, but preferably is made from a porous metal(s) that has/have surface texture due to said porosity that grips the wall surface of the facet hole and, hence, tends retain the tack in the facet hole.

FIG. 14 is a side view of another embodiment of the invented tack installed into a facet joint, wherein the bone facets are shown in cross-section. This tack comprises no barbs or protrusions formed or added to the side surface of the tack, and has a proximal end (top end) that is larger in diameter than the main body of the tack. Preferably, the tack is made from a porous metal(s) having surface texture due to said porosity that grips the wall surface of the facet hole and, hence, tends to retain the tack in the facet hole.

FIGS. 15-17 schematically illustrate some, but not the only, sequential method steps for installing embodiments of the invented tack in a spine.

FIG. 18A illustrates an example of a surface texture of one embodiment of a tack according to the invention, that is, the tack of FIG. 14.

FIG. 18B illustrates a microscopic view of the preferred porous metal(s) of which the tack of FIG. 18A is made.

FIGS. 19-24 illustrate various embodiments of fenestrated tacks according to the invention, wherein FIGS. 19-21 are side views of the tacks, and FIGS. 22-24 are top views of the tacks in FIGS. 19-21, respectively.

FIG. 25 is a cross-sectional view of the tack in FIGS. 21 and 24, viewed along the line 25-25 in FIG. 24, wherein the tack is installed on a guide-wire.

FIG. 26 is a side view of the tack of FIGS. 21, 24 installed on a guide wire.

FIGS. 27-32 illustrates alternative embodiments of fenestrated tacks, having circular apertures, wherein FIGS. 27-29 are side views, and FIGS. 30-32 are top views of the tacks of FIGS. 27-29, respectively.

FIG. 33 is a cross-sectional view of the tack in FIGS. 29 and 32, viewed along the line 33-33 in FIG. 32, wherein the tack is installed on a guide-wire.

FIG. 34 is a detail view of a portion of the core material of the tack of FIG. 33, taken from the circle marked 34 in FIG. 33.

FIGS. 35-44 are illustrations of some, but not the only, surgical method steps using percutaneous, intraoperative imaging methods and tools, which may be used to install tacks according to embodiments of the invention having longitudinal passageways through the tack body.

### DETAILED DESCRIPTION OF THE PREFERRED EMBODIMENTS

Referring to the Figures, there are shown several, but not the only, embodiments of the invented tack and methods, which are preferred for spine fixation and which may be used in various locations along the lumbar region of the spine, for example. Embodiments of the invented tacks may be used in other locations in the body, for example, wherein the tack may fix two portions of bone relative to each other or wherein the tack may be an anchor placed into a bone for attaching other structure to the bone.

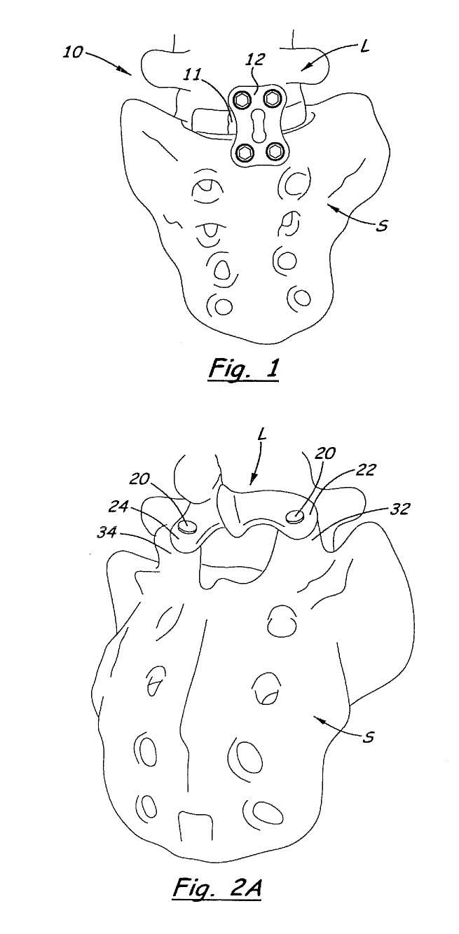

Preferably, the invented tacks are used in combination with an anterior fixation plate, such as that shown in FIG. 1. FIG. 1 is an anterior view of the lower lumbar region 10 of the spine, wherein one embodiment of an interbody implant 11 (preferably allograft), has been installed between the lowermost vertebra L and the sacrum S, and a fixation plate 12 has been installed on the anterior surfaces of said lowermost vertebra and sacrum. The rigid fixation plate 12 is screwed to said anterior surfaces, in order to fix that vertebra-sacrum joint and prevent relative movement of said vertebra and sacrum. One design of fixation plate 12, the Antegra™ device, may be obtained from Synthes, with U.S. offices in West Chester, Pa.

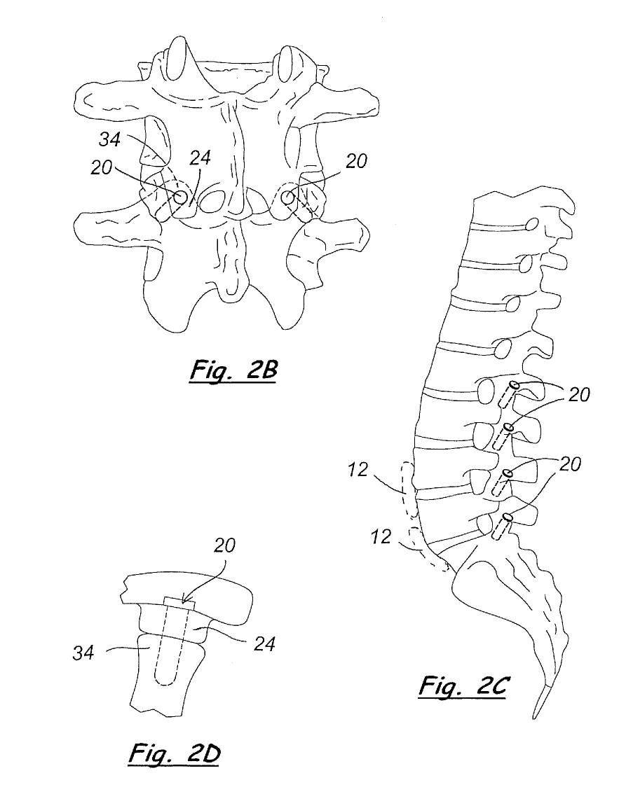

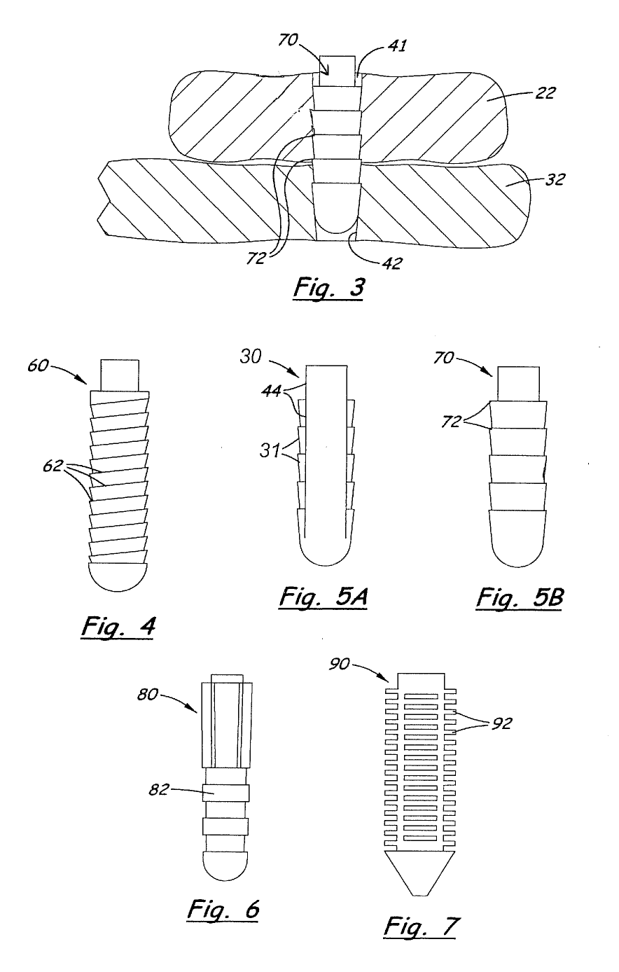

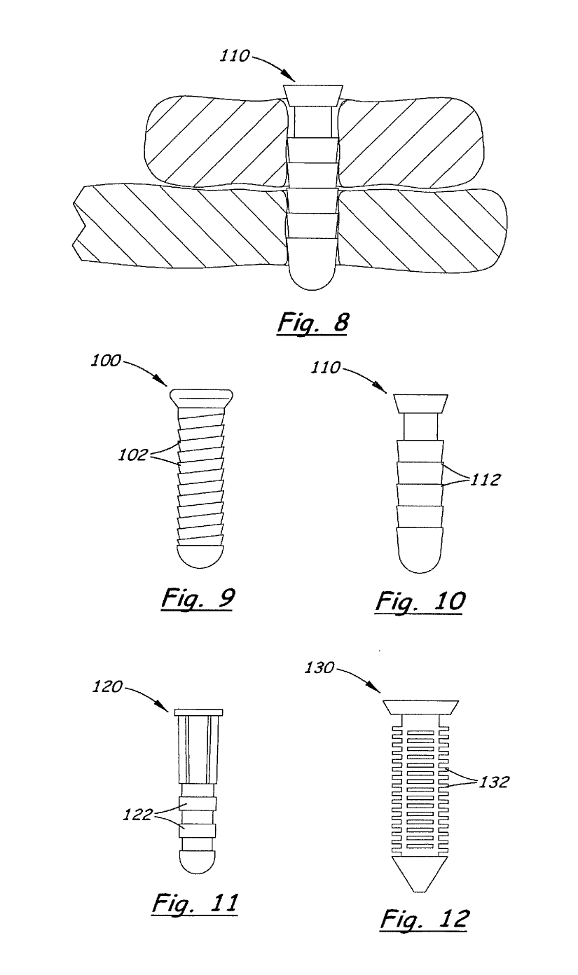

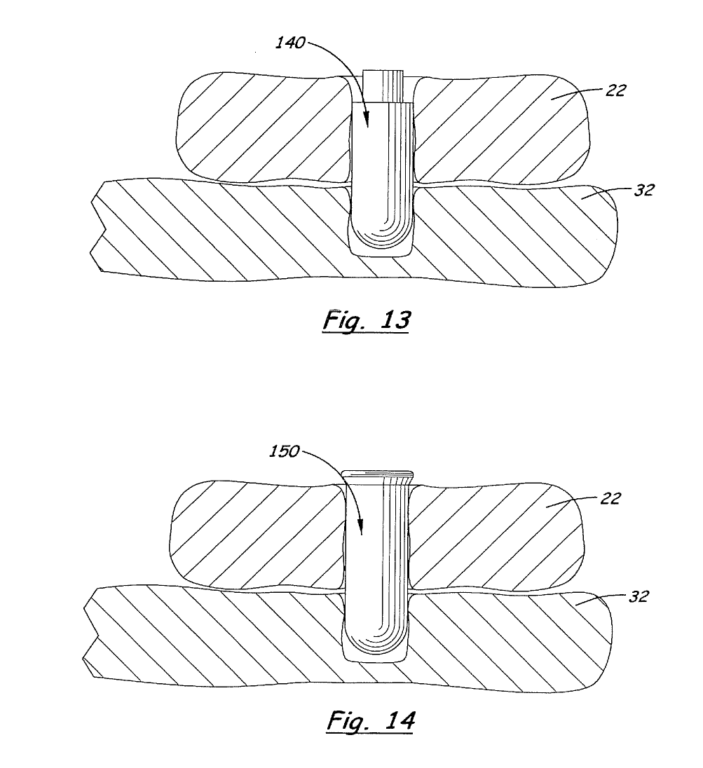

FIGS. 2A-D illustrate posterior fixation of the lumbar region 10, wherein an embodiment of the invented tack (tack 20) is used to fix inferior right and left facets 22, 24 of the lowermost vertebra L to the superior right and left facets 32, 34 of the sacrum S. Tacks 20 are installed in holes 41, 42 drilled into said facets 22, 24, 32, 34, preferably by pushing or punching the tacks 20 and not by rotating or screwing-in the tacks 20. Therefore, tacks 20 are adapted, and the methods established, for pushing into said holes 41, 42 and not screwing into said holes 41, 42. Preferably, the tacks are not rotated at all when being installed (0 degrees rotation), or, at most are rotated only slightly, for example, incidental/accidental rotation less than 10 degrees and more preferably less than 5 degrees.

Referring specifically to FIGS. 2A-D, FIG. 2A illustrates an embodiment of the invented tack the inserted into the inferior facets of the lowermost vertebra and into the superior facets of the sacrum, to fix the lowermost-vertebra-sacrum facet joint. FIG. 2B is a posterior view of two vertebrae, wherein two tacks according to embodiments of the invention are installed in (“across”) facet joints of said two vertebrae. The superior facets of the lower vertebra are fixed to the inferior facets of the upper vertebra, to prevent movement and stabilize the facets joints, and thus, to limit/prevent movement of the two vertebra to each other. FIG. 2C is a left side view of the lower spine, showing use of an embodiment of the invented tacks between the sacrum and the lowermost vertebra and also at three other locations between vertebrae. Also, in FIG. 2C, anterior plates are shown in two locations, in dashed lines, to indicate that one or more plates may be used in combination with the posterior tacks. Preferably, said anterior plates screwed or fixed to the anterior surfaces of the spine are the only anterior fixation/fusion apparatus and the posterior tacks are the only posterior fixation/fusion apparatus. It should be noted that a surgeon will not necessarily install all of the tacks shown and/or all the plates shown, but may install tacks and/or plates in one or more of these locations, for example, as needed for the particular patient. Much of the time, L4-5, and/or L5-S1 will be the subject of the fixation surgery described herein, with L3-4 and above (and all the way up to L1-2) being the subject of fixation surgery about 10-20% of the time. FIG. 2D is a detail view of a facet joint fixed by an embodiment of the invented tack.

It may be noted that there is preferably no nut, fastener, or cap for the distal end of the tack. When used to connect a first facet and a second facet, the preferred tack extends through the first facet and deep into the second facet and/or all the way through the second facet. Note that some of the Figures portray the facet hole extending all the way through the second facet and some of the Figures portray the facet hole extending part way through the second facet. There is preferably no nut, fastener, or cap that is threaded or otherwise attached on the distal end of the tack and no nut, fastener, or cap that is threaded or otherwise attached on the proximal end of the tack (except that the proximal end may be enlarged or otherwise formed for improved handling and insertion during surgery). Instead, the tack connects and “fixes” the first and second facet to each other by means of said extending through and into, and by gripping, the two facets facet but not by being fastened or “capped” at any end protruding out of the bone.

In addition, it is preferred that there is no other non-bone structure associated with, extending from, or fastened by, the tacks, so that the preferred posterior fixation apparatus consists of (closed language) the tacks and no other elements. This simple apparatus, consisting only of two preferred tacks for a set of inferior and superior facets, represents an extremely non-invasive apparatus and surgical methods for fixing the spine. The preferred tacks, therefore, are not fasteners for anchoring other non-bone elements to or around the spine, but are themselves the fixation apparatus. One may therefore differentiate the preferred tacks from bone screws that fasten other elements to the spine, such as are mentioned in the Related Art section of this document. Thus, the terminology “a posterior spine fixation apparatus consisting of one or more tacks” or “a posterior spine fixation apparatus consisting of a plurality of tacks” here and in the claims means that only the tack (preferably two tacks, a single tack in a right facet joint and a single tack in a left facet joint), with no bars, plates, extensions, supports, or other non-bone elements attached to, or extending from, the tack, is/are the posterior fixation apparatus.

Also, it is preferred that, when both anterior fixation and posterior fixation are used, that the anterior fixation is as simple and non-invasive as possible. For example, it is preferred that a simple plate, such as the plate shown in FIG. 1, is fixed to the anterior surface of the sacrum and the lowermost vertebra or to two adjacent vertebra, again with no bars, plates, extensions, supports, or other non-bone elements attached to, or extending from, the plate except that screws preferably secure the plate to said sacrum and vertebra. “Adjacent vertebrae” herein means one directly above the other in the spine. Therefore, the terminology “a spine fixation apparatus consisting of a plate fastened to anterior surfaces of two adjacent vertebrae and/or to anterior surfaces of a sacrum and an adjacent vertebra, and two tacks fixing posterior left and right facet joints . . . ” here and in the claims means that the anterior fixation device is only a plate screwed (or otherwise pinned, anchored, or fastened) to said vertebrae or said sacrum and a vertebra (and no other elements) and only two tacks, one in a right facet joint and one in a left facet joint), without additional bars, plates, extensions, supports, or other elements attached to, or extending from, the plate and the tacks. Preferred structure may also be described as “a spine fixation apparatus consisting of an interbody implant between vertebra and sacrum or between two vertebra, a plate fastened to anterior surfaces of two adjacent vertebrae and/or to anterior surfaces of a sacrum and an adjacent vertebra, and two tacks fixing posterior left and right facet joints”, which terminology will be understood by those in skill in the art after viewing FIG. 1 and reading this paragraph. Therefore, in the preferred fixation apparatus, there are no elements extending between said plate and said tacks (except that said interbody implant may be inserted as described above), no elements extending around any portion of the spine, and no elements protruding outward from the plate and/or the tacks to extend to other portions of the spine.

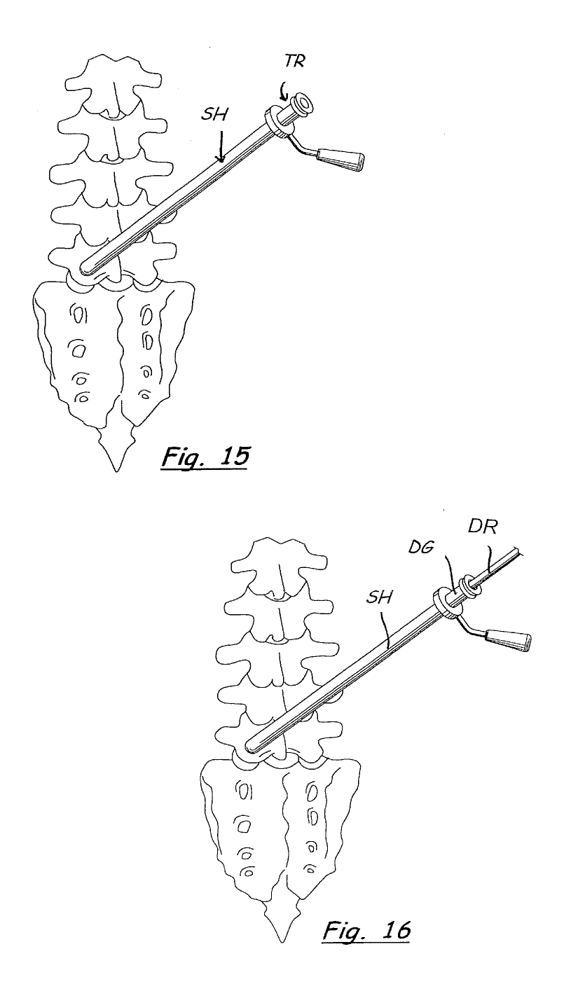

FIGS. 3-12 illustrate some, but not all, shapes in which embodiments of the invented tack may be manufacturer. Details of the tack shapes, protrusions, and upper ends are described later in this document.

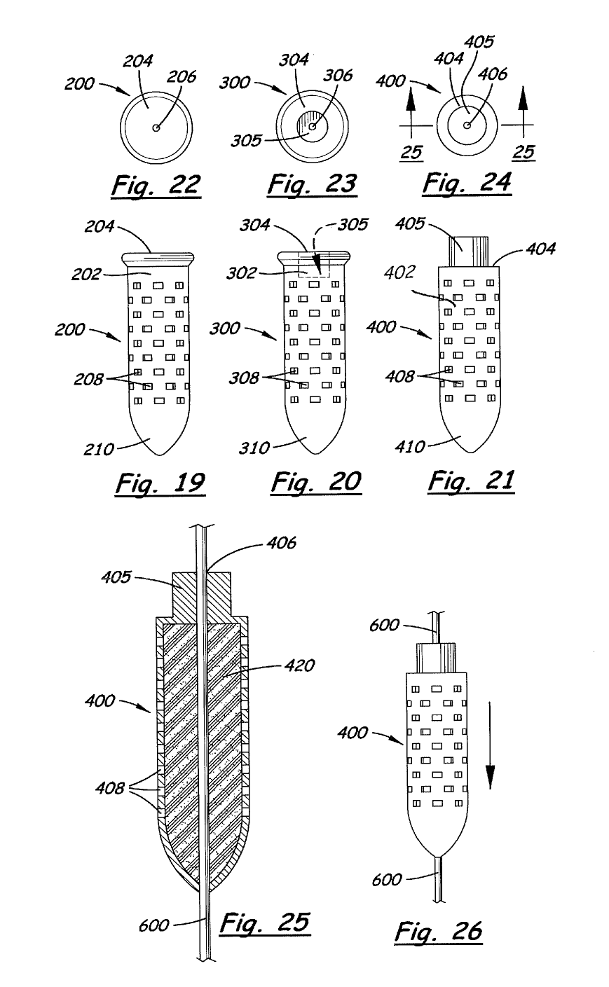

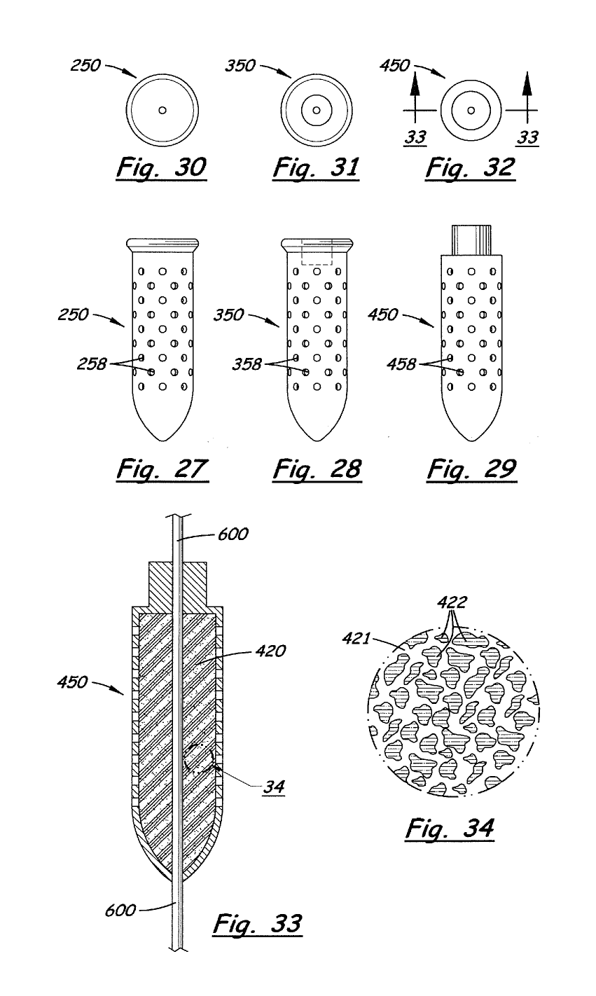

FIGS. 3 and 5B are side views of one tack 70 embodiment with protrusions 72 encircling 360 degrees around the tack axial side surface transverse to the longitudinal axis of the tack, and with a reduced diameter upper end for engagement with a tack holder. FIG. 3 shows the tack 70 installed into a facet joint, specifically into an inferior facet 22 and a superior facet 32, wherein the bone facets are shown in cross-section. FIG. 5A is a side view of another embodiment of the invented tack 30, that is similar to the tack of FIGS. 3 and 5B, but with protrusions 31 extending from left and right sides of the tack body 44 and not extending from locations 360 degrees around the tack.

FIG. 4 is a side view of another embodiment of the invented tack 60, wherein the protrusions are intended to abut/grip the wall surface of a hole drilled across the facet joint. The protrusions 62 in this embodiment are slanted relative to the transverse (radial) dimension of the tack, but said protrusions are not meant to be threads. The protrusions are merely for abutting/gripping the wall surface upon axial insertion of the tack into the hole, and the tack is preferably not rotated into the hole.

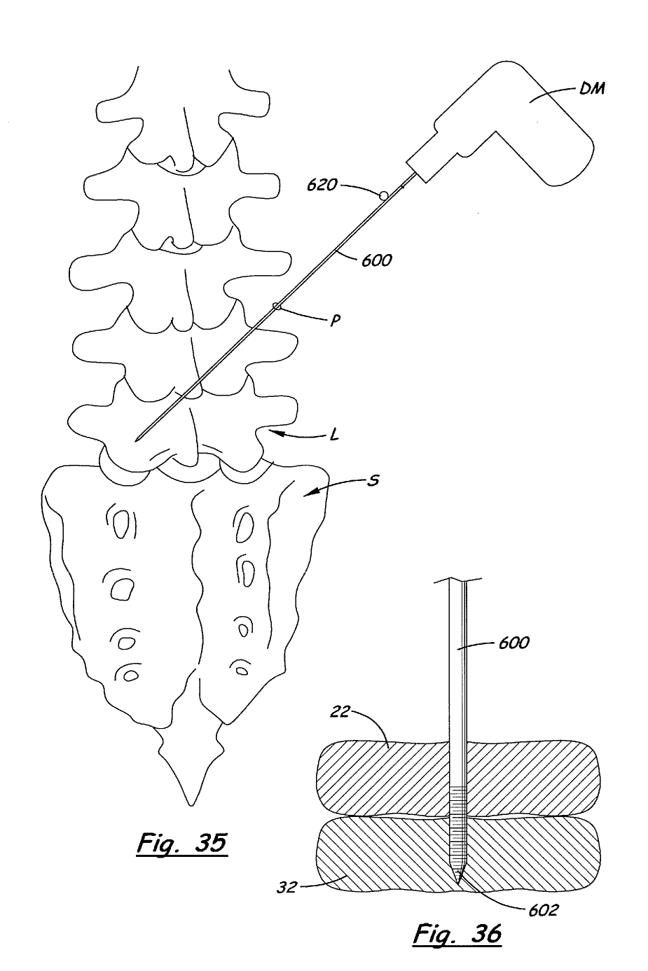

FIG. 6 is a side view of another embodiment of the invented tack 80, wherein the (bottom) tip of the tack is rounded, protrusions 82 extend from the side surface of the tack near the tip, and axial slots are provided near the top end of the tack.

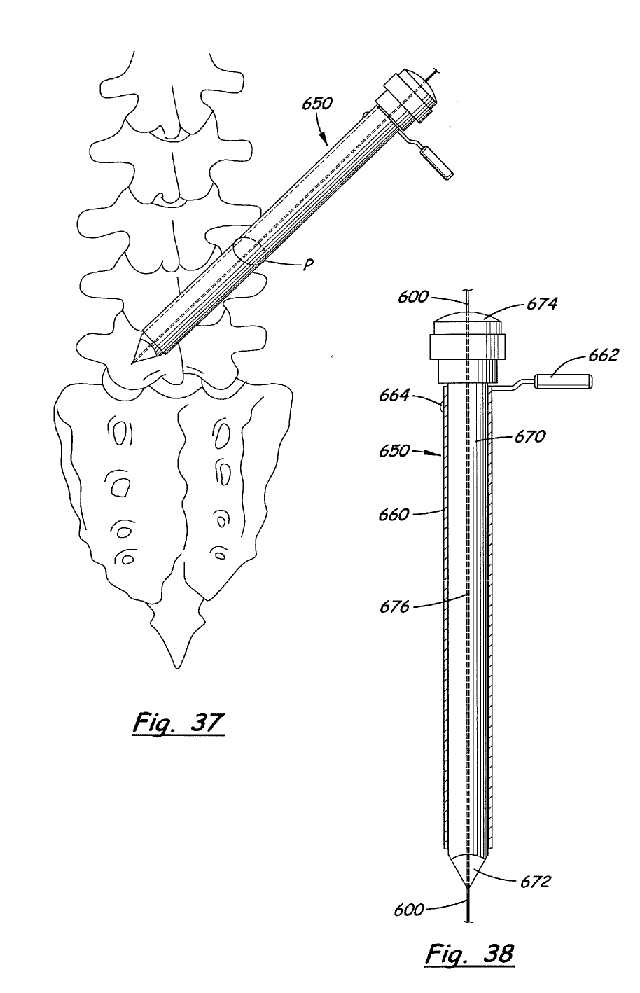

FIG. 7 is a side view of another embodiment of the invented tack 90, wherein multiple, shelf-like protrusions 92 extend from the tack axial side surface in four locations around the tack (for example, spaced generally 90 degrees).

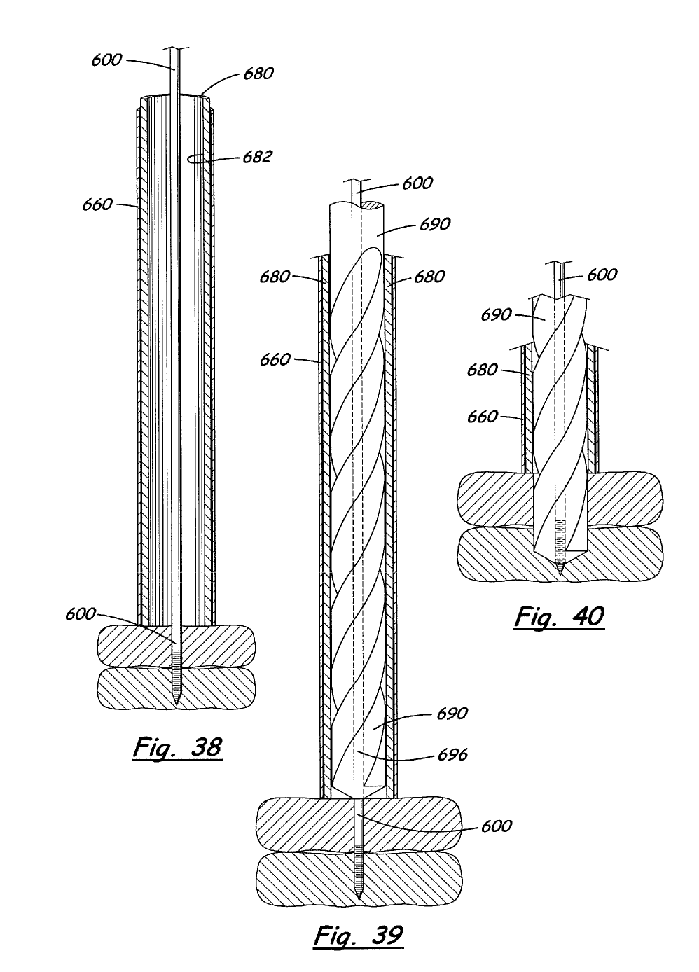

FIG. 8 is a side view of another embodiment of the invented tack installed into a facet joint, specifically into an inferior facet and a superior facet, wherein the bone facets are shown in cross-section. This tack is similar to that shown in FIGS. 3 and 5B, but comprises an enlarged top end.

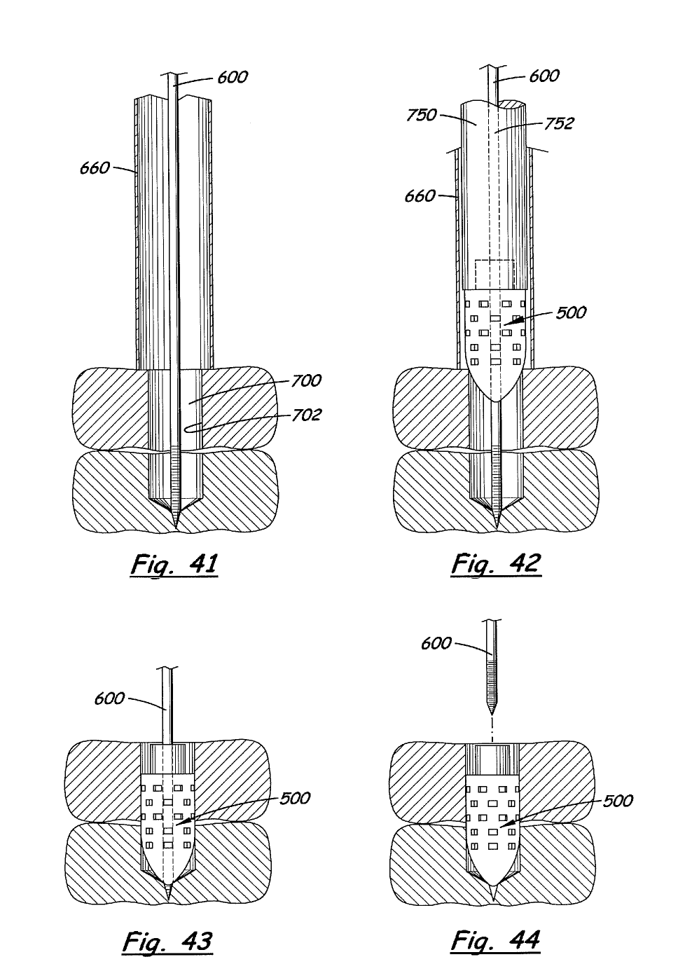

FIGS. 8-12 portrays alternative embodiments of the invented tack, wherein the upper end of each tack is enlarged. Tack 110 with protrusions 112 in FIGS. 8 and 10 is similar to tack 70 in FIGS. 3 and 5B, tack 100 with protrusions 102 in FIG. 9 is similar to tack 60 in FIG. 4, tack 120 with protrusions 122 in FIG. 11 is similar to tack 80 in FIG. 6, and tack 130 with protrusions 132 in FIG. 12 is similar to tack 90 in FIG. 7, except for the enlarged upper end that may be adapted for engagement with a tack holder and/or impact tool. As discussed above for the embodiment of FIG. 4, tack 100 of FIG. 9 has protrusions 102 are slanted relative to the axial dimension of the tack, but said protrusions are preferably not used as threads for threaded rotational insertion. The protrusions 102, 112, 122, 132 are intended to abut/grip the wall surface of a hole drilled across the facet joint upon axial insertion of the tack into the hole, but the tack 100, 110, 120, 130 are preferably not rotated into the hole.

FIG. 13 is a side view of another embodiment of the invented tack 140 installed into a facet joint, specifically into an inferior facet 22 and a superior facet 32, wherein the bone facets are shown in cross-section. This tack main body is cylindrical in shape with a rounded bottom end, and has with a smaller-diameter cylinder protruding upward from the main body of the tack to create a radial shelf, at the junction between the larger-diameter and smaller-diameter portions of the tack. The top end (also “proximal end”) that is smaller in diameter than the main body of the tack and said radial shelf may be used for engagement and/or impacting by the tool that is used to axially-force the tack into the facet hole. This tack comprises no barbs or protrusions formed or added to the side surface of the tack. Preferably, the tack is made from a porous metal(s), as discussed in more detail below, which has surface texture due to said porosity that grips the wall surface of the facet hole and, hence, tends to retain the tack in the facet hole.

FIG. 14 is a side view of another embodiment of the invented tack 150 installed into a facet joint, specifically into an inferior facet 22 and a superior facet 32, wherein the bone facets are shown in cross-section. This tack comprises no barbs or protrusions formed or added to the side surface of the tack, and has a proximal end (top end) that is larger in diameter than the main body of the tack. This tack main body is cylindrical in shape with a rounded bottom end. The top end (also “proximal end”) forms a slightly-enlarged (relative to the main body) radial shelf or radial surface that may be used for engagement and/or impacting by the tool that is used to axially-force the tack into the facet hole. Optionally, an indentation may be provided in the top end of this tack, for example, at the axial centerline, to assist in said engagement/impacting by said tool. This tack comprises no barbs or protrusions formed or added to the side surface of the tack. Preferably, the tack is made from a porous metal(s), as discussed in more detail below, which has surface texture (FIGS. 18A and B) due to said porosity that grips the wall surface of the facet hole and, hence, tends to retain the tack in the facet hole.

FIGS. 15-17 schematically illustrate some, but not the only, methods of installing embodiments of the invented tack in a spine. FIG. 15 schematically illustrates a method of docking the trocar TR and sheath SH on the medial facet of the joint to be stabilized. FIG. 16 schematically illustrates a step wherein a drill guide DG and drill DR may be inserted into the sheath, and the drill is advanced through the midportion of the medial facet across the facet joint. FIG. 17 schematically illustrates a tack holder TH, holding a tack according to embodiments of the invention, being placed through a sheath for impacting across the facet joint, for example, by a mallet MT. Various trocar, sheath, drill guides, drills, tack holders, and impact tools may be used for the insertion and location, followed by impacting, of the tack into the facet hole, as will be understood by those of skill in that art after reading and viewing this disclosure.

FIG. 18A illustrates an example of a surface texture of one embodiment of a tack 150 according to the invention, that is, the tack of FIG. 14, which is a rough/uneven texture resulting from porosity of the material from which the tack is made. At least the sidewall of the tack, preferably 360 degrees around the tack, has this desirable texture, and, typically, all of the exterior surface of the tack has this desirable texture.

FIG. 18B illustrates a microscopic view of the preferred porous metal(s) of which the tack of FIG. 18A is made, which results in a texture that is adapted for excellent gripping of the facet hole wall surface and adapted for excellent bone growth into the porous structure of the tack after the tack has been installed.

One may note from FIGS. 1-18 that various upper end (“proximal end”) shapes may be provided on the tack. For example, an enlarged end or “tack head” in some embodiments may be provided, such as in the examples in FIGS. 2 and 8-12, 14, and 18A. Or, for example, the proximal end of the tack may not be enlarged relative to the main body of the tack or may even be of smaller diameter than the main body of the tack, for example, see FIGS. 3-7. Thus, it is not necessarily required to have an enlarged proximal end, or other enlarged cap or fastener on the proximal end of the main body. While various proximal end designs and cooperating tack holders/impact-tools may be designed, the smaller diameter proximal end may be useful as a means for a hollow-ended tool to surround and capture said proximal end for sure placement of the tack into the patient, preferably through a sheath. Also, this capture of the proximal end of the tack may allow the tack to be impacted (through the tool) without the tool sliding off of the tack, and without the tool gouging or chipping the bone or adjacent soft tissue or otherwise enlarging the hole in the bone during the impact. After the initial drilling of the hole, the tool(s) used to impact/install the tack preferably do not need to impact or touch the bone.

The tacks preferably comprise bioactive material that promotes/accepts bone growth either by virtue of the bioactive material having pores that match or accept natural bone growth or by virtue of being made of material that is naturally replaced by growing bone, for example, in “resorption” or absorption” of the bioactive material and replacement of it by growing bone. The preferred tacks shown in FIGS. 2A-18A preferably are entirely or substantially entirely made of material that encourages or receives bone growth, or have at least an outer housing or surface made of said material that encourages or receives bone growth. Preferred materials are bioactive material that will be in contact with the facet bone upon installation of the tack. The tacks in their entirety or their outer housing or surface may be made from one or more of the following materials, for example: machined allograft, beta-tricalcium phosphate polymer, and porous tantalum or other porous metal or metal composite materials, stainless steel, titanium, carbon fiber, PEEK (polyether ether ketone) and other polymers. Special treatment of the material or at least the tack external side surface, such as formation of texture or pores that promote bone growth into the textured/porous surface, may be used for materials that are inherently smoother than desired. Later in this document are described tack embodiments wherein the bioactive material is provided in a core of the tack that is contained within a fenestrated housing of other materials.

The preferred materials used for the entire tack, or the outer surface or housing, are strong in the axial direction, so that the tacks may withstand the impact/force of being pushed/impacted into said holes 41, 42, even when the tacks are made to be very small (for example, 4-6 mm in diameter). It is important that the tacks be made to be very small in order to fit into/through the facets 22, 24, 32, 34, which are small bone portions protruding out from the vertebrae and sacrum, as is well know in the medical arts. Multiple sizes of tacks may be made, for example, a small tack with main body of 4.0 mm diameter (also called “inner diameter”) and protruding out 0.5 mm, for example, for an outermost tack diameter of 4.5 mm (also called “outer diameter”). A medium tack may be made with 4.5 mm main body diameter and 0.5 mm protrusions for an outermost tack diameter of 5.0 mm. A large tack may be made with 5.0 main body diameter and 0.5 mm protrusions for an outermost tack diameter of 5.5 mm. An extra large tack may be made with 5.5 mm body diameter and 0.5 mm protrusions for an outermost tack diameter of 6.0 mm.

Holes are drilled, and the tacks chosen, for a close fit, and preferably even a tight fit (but not risking breakage of the tack or the bone), between the hole wall (bone surface) and the tack generally cylindrical side surface and/or it protrusions. The tacks, preferably, do not bend or deform a significant amount, when impacted/forced into the hole, except, for example, deformation of portions of the axial side surface of the tack main body and/or protrusions therefrom on the order of approximately 0.1-1 mm, as further described below in order for a tight fit to be obtained.

It is preferred that the tacks have main body surfaces (axial sidewall diameter) of less than or equal to 6 mm, and, more preferably, in the range of 4-6 mm. For example, in especially-preferred embodiments, cylindrical hole of 4.5 mm diameter is drilled through an inferior facet and into a corresponding superior facet. Then, a 5 mm tack is installed in the hole, wherein the main body largest diameter is 5.0 mm but the protrusions may extend out to increase the diameter by 0.5 min diameter, for a outermost outer diameter of about 5.5 mm. Thus, this slightly larger-than-the-hole tack diameter will typically represent the main body being slightly larger than the hole (5.0 mm diameter main body not counting the protrusions) and the diameter of the protrusions of the tack being even larger (5.5 mm diameter overall counting the protrusions), so that forcing of the tack into the hole may deform the tack protrusions and possibly even the main body slightly, and/or may deform the bone hole surface slightly so that the tack becomes tightly installed in the hole and unlikely to “back out” of the hole. In the event that the hole has not been formed accurately-enough during drilling by the surgeon, and/or the tack or bone deforms too much to create a tight fit, a “salvage tack” of a larger diameter may be used, for example, a 5.5 mm diameter tack (5.5 mm at its largest diameter not counting the protrusions, for a total of approximately 6 mm with protrusions) to be installed in and securely remain in the inaccurate, nominal 4.5 mm hole.

In embodiments with no protrusions, such as are represented in FIGS. 2D, 13, and 14, 17, and 18A, the cylindrical diameter of the tack is preferably close to the diameter of the facet hole. For example, a 4.5 mm hole is drilled and the tack has an outer diameter (preferably consistent or nearly consistent all along the length of its main body) of 4.5-5.0 mm, with a “salvage tack” having a diameter of 5.0-5.5 mm, for example. Depending on the accuracy of the hole formation and the precision of the tack diameter, a closer fit (4.6-4.8 mm for the tack to be secured held in a 4.5 mm hole) may be possible. It may be understood that various tack diameters may be effective for various hole sizes and drilling procedures, especially in view of the materials of manufacture of the tacks, and this will be determinable by one of skill in the art without undue experimentation.

The preferred bioactive materials are expected to be relatively brittle upon torsion, and, especially brittle when the tack is made to be very small (4-6 mm). Therefore, the preferred tacks, especially those in which at least the outer surface/housing are bioactive materials, are intended to be pushed or axially-impacted only, and not rotated or otherwise subjected to torsion. Therefore, while barbs or other protrusions may be provided on the outer axial surfaces of a first group of tack embodiments, it is preferred that these barbs/protrusions are not adapted to encourage or cause rotation of the tack in the holes and it is preferred that these barbs/protrusions are strong enough so that they do not snap or otherwise break when being installed, even if there is said incidental/accidental rotation. Therefore, while protrusions, such as the slanted protrusions in FIGS. 4 and 9, may be acceptable in some embodiments, it is preferred that such slanted protrusions do not extend continuously around the main shaft of the tack and/or that the tack is purposely not rotated during installation and so no attempt is made to screw-in the tack.

The preferred posterior fixation system consists only of two tacks according to embodiments of the invention, and no additional bars, plates, arms, or hooks attached to the preferred tacks or on the posterior side of the lumbar region. Thus, although a portion of the upper end of the tack may, in some embodiments, protrude out from the bone, preferably 90 percent or more of the apparatus for anterior fixation is installed inside/within the bone and does not protrude or lie along outer bone surfaces. Thus, the simplicity of the preferred apparatus and the minimally-invasive methods of installing the preferred apparatus provide benefits during and after surgery.

Each of the illustrated tacks 20, 30, 60, 70, 80, 90, 100, 110, 120, 130, 140, 150 may be described as an elongated member, which may also be called a non-threaded pin or anchor because the tacks have no threads. Each of the tacks has a main body surface that is generally cylindrical along its entire length or, at least generally cylindrical along the portion of the tack that extends into the facets.

Tacks 30, 60, 70, 80, 90, 100, 110, 120, 130 may be called a first group of tack embodiments that comprise protrusions 31, 62, 62, 72, 82, 92, 112, 122, 132 extending from said main body surface, wherein some or all of said protrusions are radially-extending or directed toward the top end so that they do not significantly interfere with insertion of the tack into the holes, and so that they do interfere with the tack backing up out of the holes during and after surgery. In other words, it is preferred that the protrusions do not extend in a direction toward the distal (lower) end of the tack.

In the second group of embodiments, the main bodies of tacks 20, 140 and 150, however, are generally cylindrical and do not have protrusions. The main bodies of tacks 20, 140 and 150 have sufficient texture, preferably of very small scale such as a rough and/or porous surface, that the main body tends to grip the bone surface of the hole into which it is impacted and resist or prevent the tack from backing up and out of the hole. In the case of the preferred bioactive materials, even a small amount of bone growth into the bioactive material will further increase the interaction and stability of the tack in the bone, so that the tack, over time, becomes even less likely to reverse itself out of the hole/facet. Barb-less and protrusion-less tacks 20, 140, 150 do not have internal spaces or hollow regions other than the void space caused by the porosity of the material of the tack, but fenestrated embodiments described below have apertures into the outer surface of the tacks. Barb-less and protrusion-less tacks may have a recess (not shown in FIGS. 1-18), or other engagement structure on its proximal end that may cooperate with a trocar or other sheath, or other placement or impact tool, during installation of the tacks in the human body.

Preferred materials for tack 140 and 150, and other protrusion-less or barb-less tacks, include machined allograft, beta-tricalcium phosphate polymer, and porous tantalum, porous-tantalum-containing, or other porous metal or metal composite materials, stainless steel, titanium, carbon fiber, PEEK (polyether ether ketone) and other polymers. Special treatment of the material or at least the tack external side surface, such as formation of a texture or pores that promote bone growth into the textured/porous surface, may be used for materials that are inherently smoother than desired. Porous tantalum metal is of particular interest for such tacks, because it is believed by the inventor to have a surface texture/porosity well-adapted for bone growth into said texture/porosity. One example of a porous tantalum material/composite is Zimmer Trabecular Metal™ (see Zimmer.com and/or U.S. Pat. No. 5,282,861, which patent is hereby incorporated in its entirely into this disclosure by this reference). Trabecular Metal™ is reported to be elemental tantalum metal material formed by vapor deposition techniques that create a metallic strut configuration similar to trabecular bone. Later in this document are described tack embodiments wherein bioactive material is provided in a core of the tack that is contained within a fenestrated housing of other materials.

One may see in the figures the preferred locations and methods of installation of the invented tacks. The lumbar region of the spine is the particularly-preferred, but not necessarily the only, location for use of the invented tacks. Many patients with discogenic back pain have canal pathology (e.g., stenosis, migrated HNP) that requires posterior decompression, which has in the past prompted, and continues to prompt, many surgeons to choose an all-posterior approach with TLIF/PLIF and pedicle screw instrumentation which results in a large incision, more blood loss, paraspinal muscle denervation and increased risk of “fusion disease”. With the development of modern, stand-alone anterior lumbar plates (such as plate 12 in FIG. 1), minimally invasive anterior lumbar interbody fusions have become an attractive treatment option for the treatment of discogenic back pain. However, many surgeons have concerns with stand-alone anterior constructs (anterior constructs as the sole, only interbody fusion apparatus and methods), especially in the setting of previous or concurrent midline decompression with loss of the posterior ligamentous tension band. Therefore, the inventor believes that his facet fixation tack device designed specifically for lumbar facet immobilization and fusion will perform extremely effectively in combination with an anterior fusion apparatus.

Therefore, an especially-preferred combination for the lower spine, as portrayed to best advantage in FIGS. 1 and 2A, is an anterior fixation plate, an interbody allograft insert (as will be understood by those of skill in the art, given this disclosure), and two tacks according to embodiments of the invention, wherein the two tacks serve to fix the lowermost vertebra right and left facets to the sacrum superior right and left facts, respectively. Thus, the preferred device provides immediate immobilization of the facet joint in order to augment anterior plating, and is biologically active and facilitates fusion of the facet joint. Thus, the preferred tacks are designed to be utilized as a minimally invasive adjunct to anterior lumbar interbody fusion procedures, and provide unique immobilization of facet joints with bioactive materials that preferably eventually become incorporated into a durable facet fusion.

The preferred tacks comprise the additional benefit and feature of being easily inserted through a posterior “microdiscectomy incision”. Thus, the especially-preferred embodiments are a fixation and fusion device designed to be utilized as a minimally-invasive adjunct to anterior lumbar interbody fusion procedures. The preferred lumbar facet fixation device is designed to provide initial immobilization of the facet joint with bioactive materials that eventually become incorporated into a durable facet fusion. In many barbed embodiments, the cylindrical tack device will have an “inner” diameter (main body exterior diameter, not counting barbs/protrusions) of 5.0 mm, and a tapered or rounded leading surface to facilitate insertion. Barbs provided around the shaft (main body) may create an outer diameter (outermost diameter, counting barbs/protrusions) of 5.5 mm, for example. The lengths of the tacks are expected to range from 10 mm to 30 mm in 2 mm increments, for example. The “salvage” tacks, as discussed above, are expected to be approximately 5.5 mm inner diameter (not counting barbs/protrusions) and 6.0 mm outer diameter (counting barbs/protrusions).

As may be seen schematically in FIGS. 15-17, the preferred methods of installation comprises a bilateral mini-laminotomy incision (not shown but understood in view of the figures by one of skill in the art), followed by a sheath SH and trocar TR placed percutaneously and docked on medial facet. A 5.0 mm diameter drill DR may be advanced through midportion of the medial facet across the facet joint, exiting the lateral facet with a trajectory of dorsosuperomedial to anterioinferolateral. A tack of appropriate length is selected and may be inserted part way into the resulting hole in the inferior facet (herein called the “facet hole” or “holes”), due to the preferred tapered/rounded leading end of the tack, and then impacted across the facet joint, for example, by various placement tools, such as a tack holder envisioned by the inventor, and impacting tools, including a mallet MT or other means of providing force. One of skill in the art, upon reading and viewing this document and the Figures, will understand that various styles of preferably-minimally-invasive tools may be used for the installation steps or drilling, installing and impacting, to create a tight fit between the tack and the bone, wherein, as discussed above, the preferred installation steps do not comprise rotation of the tack upon its longitudinal axis. Once one of the preferred pair of tacks is installed, the steps are repeated across the contralateral facet joint.

A specific example of insertion techniques for the especially-preferred embodiments is as follows. In a minimally-invasive approach, if a midline decompression is required, a mini-laminotomy-incision is made in the midline exposing the posterior elements to the area of the medial facet joints bilaterally. A percutaneous stab incision is made superior and contralateral to the facet joint to be fixed with the tack. A sheath and trocar are then inserted through the stab incision between the interspinous ligaments or dorsal to the spinous processes. The sheath is docked on the medial facet of the joint to be stabilized. A drill guide is inserted into the sheath and a 5 millimeter drill is advanced through the midportion of the medial facet across the facet joint exiting the lateral facet with a projectory from dorsosupermedial to aneroinferolateral. The length of the tack required for the particular patient and the particular facet joint may be read directly off the drill guide, or alternatively may be measured with a depth gauge placed through the sheath. A tack guide is then placed through the sheath and an appropriate length tack is placed through the guide and impacted across the facet joint. This is then repeated in an identical fashion across the contralateral facet joint. It is recommended that the tack be placed prior to any lateral decompression to minimize the risk of facet fracture. If a midline decompression is not required, this technique can be utilized through two small muscle splitting incisions placed directly over the involved facet joints using the same percutaneous drill and tack technique. Alternative methods, and/or incision locations and projectories, may be used, depending on the patient, the patient's particular injury or spine damage, whether laminectomy is needed, and/or other issues; these issues will be understood by those in skill in the art and may be addressed without undue experimentation. Further description and portrayal of surgery methods, for use with guide-wire and intraoprative image techniques, are included later in this document under Especially-Preferred Embodiments.

### Especially-Preferred Embodiments

FIGS. 19-34 illustrates especially-preferred, fenestrated tack embodiments that comprise a fenestrated outer housing, a biologically active core material(s), and preferably a longitudinal passageway for receiving a guide-wire used in surgical methods. Tacks 200, 300, and 400 all comprise a hollow housing 202, 302, 402 with various types of upper ends for engagement by a tack holder or other insertion/impacting tools. Tack 200 has a slightly-enlarged upper end with a radial top surface 204 and guide-wire hole 206 extending all the way from the top surface 204 through the tip of the tapered distal end 210, coaxial with the longitudinal axis of the tack. Housing 202 comprises rectangular apertures 208 rectangular apertures through the housing wall into the hollow interior space of the housing. Tack 300 also has a slightly-enlarged upper end with a radial top surface 304, but this tack has a recess 305, coaxial with the longitudinal axis of the tack and the guide-wire hole 306, for cooperating with a tack holding tool and/or impact tool. The guide-wire hole 306 extends all the way from the bottom of the recess 305 through the hollow interior space of the tack, and through the tip of the tapered distal end 310. Housing 302 also comprises rectangular apertures 308 rectangular apertures through the housing wall into the hollow interior space of the housing. Tack 400 has radial top surface 404, with a cylindrical body 405 upending from the top surface 404, coaxial with the longitudinal axis of the tack and with the guide-wire hole 406, for cooperating with a tack holding tool and/or impact tool. As the tack is preferably not rotated during insertion into the facets, the preferred holding tool need not engage in such a way that the holding tool could rotate the tack on longitudinal axis, and, hence, a cylindrical body 405 being engaged by a cylindrical recess in the distal end of the holding tool may be effective. The guide-wire hole 406 extends all the way from the bottom of the recess 405 through the hollow interior space of the tack, and through the tip of the tapered distal end 410. Housing 402 also comprises rectangular apertures 408 rectangular apertures through the housing wall into the hollow interior space of the housing.

The housings 202, 302, 402 are preferably made of metal(s) such as titanium or stainless steel, but may be other metals, polymers, and/or composites capable of withstanding the conditions of sterilization and the forces of insertion. Inside the hollow interior space of the housings 202, 302, 402 is provided one or more biologically-active materials. Once the tack 200, 300, 400 is installed in the facet joint, bone growth will occur through the apertures 208, 308, 408, and into the material inside the tacks. For example, the material provided inside the tacks may comprise one or more of osteobiologic material, demineralized bone matrix (DBM), sponge holding bone morphogenic protein (BMP, a bioengineered protein), allograft and/or other materials. The apertures 208, 308, and 408 are preferably 0.1 mm up to 1 mm in diameter (or in both length and width), for example, while pores for a porous metal such as tantalum may be on the order of micrometers, for example, 400 micrometers in diameter. Apertures may be other shapes, for example, oval, triangular, pentagonal, hexagonal, octagonal, and others.

FIG. 25 shows the tack 400 in cross-sectional view, wherein the wall of the housing substantially surrounds an interior space that contains core 420, which preferably comprises biologically-active material(s) that receive(s) and/or encourage(s) bone growth. FIGS. 25 and 26 also illustrate the guide-wire 600 extending through the tack 400, wherein the tack is slidable along the guide-wire as is described in more detail below.

FIGS. 27-34 further illustrates embodiments of fenestrated tacks, which are tacks 250, 350, 450 with circular apertures 258, 358, 458. The upper ends of the tacks are similar to those described regarding the tacks 200, 300, 400 in FIGS. 19-26. Tacks 250, 350, and 450 also each have a longitudinal passageway coaxial with the longitudinal axis of the tack, adapted to receive and slide along a guide-wire 600. The material of the core material inside the hollow housings may comprise one or more biological-active materials that receive and/or encourage bone growth, such as such as osteobiologic material, demineralized bone matrix (DBM), sponge holding bone morphogenic protein (BMP, a bioengineered protein), allograft and/or other materials. Tack 450 is shown with core 420, which is a sponge material 421 containing/holding bone morphogenic protein 422 (BMP, a bioengineered protein). See FIG. 34. After insertion of the tack 450 across the facet joint, bone growth will begin, extend through the apertures 458 and into the core 420, encouraged/enhanced by the BMP 422. The apertures 458 are preferably in the range of 0.1-1 mm, and are expected to allow substantial bone growth through the wall of the housing and through the core, to form a permanent fixation of the two facet bones by bone extending between both facets through the tack.

FIGS. 35-44 illustrate one set of, but not the only, method steps for using embodiments of the invented tack that comprise a longitudinal passageway for receiving and sliding on a guide-wire. These and other steps may be used in percutaneous surgery with intraoperative image guidance, in order to minimize invasiveness of the surgery. Percutaneous surgery using intraoperative image navigation are known to those of skill in the art, who will understand from this disclosure and the drawings how to use embodiments of the invention in such surgery. Various companies provide imaging and equipment for such surgery, for example, Medtronic Sofamor Danek provides the O-Arm™ system.

In the preferred surgery methods, after reference-point equipment is installed in/on the patient in another area of the spine, a guide-wire is inserted into the patient by means of the guide-wire 600 being pushed and/or drilled by a hand-held drill motor DM. The guide-wire 600 is a small-diameter, rigid wire with a sharp or drill-like distal end 604. The wire 600 percutaneously enters the patient through the skin (at P in FIG. 35), thus reducing or eliminating the need for an incision. The wire 600 is drilled across the facet joint, as shown in FIG. 36 and remains there until near the end of surgery. The wire 600 establishes a line extending from outside the patient's body to the site of tack installation, and serves as an elongated member along which tools and the tack may be slid to said site coaxially with the wire. The wire has an LED or other signal or transmitting device 620, which the interoperative imaging navigation system uses to relate to the reference-point(s) to provide images to guide the surgeon. In all the subsequent steps and with the subsequent tools, the imaging navigation system will guide the surgeon by monitoring the surgical tool location by means of such LED or other signal or transmitting devices 620, 664.

In FIG. 37, a sheath and trocar assembly 650 are inserted through the incision, thus making the opening in the skin (see P in FIG. 37) larger, but still far smaller than traditional incisions. The sheath 660 is generally a hollow, open-ended cylinder into which the trocar is received coaxially. The trocar 670 has a longitudinal passageway 676 (is “cannulated”) just slightly larger in diameter than the wire 600, so that the trocar, and hence the assembly 650, can be slid onto and along the guide-wire 600. The sheath 660 and trocar 670 are both coaxial with the wire 600, and may be advanced inside the patient to the site, utilizing the sharp end 672 of the trocar to open/spread the soft tissue. Thus, in use, the guide-wire extends all the way through the sheath and trocar assembly 650, from the sharp end 672 of the trocar to the top end 674 of the trocar. The sheath 660 typically has a handle 662 for manipulation of the assembly 650.

After removal of the trocar 670 from the sheath, a drill guide 680 is slid into the sheath 660, over the wire 600, as shown in FIG. 38. The drill 690 is then inserted into the drill guide, coaxial with and over the wire 600, as the drill 690 has a longitudinal passageway 696 that can receive and slide along the wire 600, as shown in FIG. 39. The drill guide inner surface 682 has a diameter only slightly larger than outer diameter of the drill 690. The drill 690 is advanced to the site inside the drill guide 680, and used to drill a hole 700 across the facet joint for receiving the tack, as shown in FIGS. 40 and 41. As shown in FIG. 41, removal of the drill guide 680 and drill 690 reveals hole 700 that has a hole surface 702 that is slightly smaller diameter than the inner diameter of the sheath 660. Also as shown in FIG. 41, the wire 600 as originally installed is deeper in the bone than the hole 700, so the wire 600 can remain in the bone after drilling of the hole 700 and removal of the drill.

The tack 500 is then installed on the guide-wire 600, by means of the tack longitudinal passageway receiving and sliding along the wire 600. As shown in FIG. 42, the tack is advanced toward the hole 700 by a tack holder 750 also having a longitudinal passageway 752 (“cannulated”) that receives and slides along the wire 600. The holder 750 may have a distal end adapted to receive and engage a portion of the upper end of the tack. An impact tool (now shown in this series of figures) is used to force the tack into the hole, into a position extending across the facet joint, and the holder 750 and sheath 660 are removed from the patient, as shown in FIG. 43. The wire 600 may then be “unscrewed” or otherwise removed from the bone and removed from the patient, as shown in FIG. 44, leaving the tack 500 in place. It is in this position inside the patient that bone growth begins soon after surgery, to enter the apertures of the housing of the tack and grow into the core of the tack.

It will be understood that the dimensions of the preferred longitudinal passageways extending through the tacks, trocar, drill, and tack holder will each be of diameter slightly larger than the outer diameter of the guide-wire, which guide-wires are commercially available and known in the surgical arts. Examples of passageway diameter for the preferred 4-6 mm diameter tacks may be a fraction of a millimeter up to 3 millimeters in diameter, for example.

Alternative embodiments of the invention may include a longitudinal passageway through the tack for receiving and sliding along a guide-wire, wherein the tack is a solid tack without a hollow interior space. Such embodiments may comprise adding the wire passageway to tack bodies similar to those in FIGS. 2-18, for example, solid bodies with porous or textured body or sidewall and/or protrusions. In embodiments without the biologically-active core, it is preferred that the porous/textured body or sidewall have biologically-active features that will receive and/or encourage bone growth.

Further, other embodiments may be of the hollow-housing-with-core design and not be adapted to cooperate with a guide-wire, or simply not be used with a guide-wire. Therefore, some tacks may have the hollow-housing-with-core design, for example, wherein biologically-active materials are in the core, while not having the longitudinal passageway for the guide-wire. Such embodiments may optionally have protrusions extending radially outward from the sidewall of the tack housing, for example around or inbetween the apertures that extend from the outer surface of the sidewall to the interior surface.

Other embodiments of the invented apparatus and methods will be apparent to one of skill in the art after reading this disclosure and viewing the drawings. Although this invention is described herein with reference to particular means, materials and embodiments, it is to be understood that the invention is not limited to these disclosed particulars, but extends instead to all equivalents within the broad scope of the following claims.

1. A posterior spine fixation apparatus comprising a tack comprising a hollow housing with an interior space, and core material received inside the interior space, wherein said hollow housing has a side wall comprising apertures extending from an outer surface of the side wall to the interior space, said core material comprising biologically-active material.

2. The posterior spine fixation apparatus as in claim 1, wherein said biologically-active material is selected from a group consisting of: osteobiologic material, demineralized bone matrix (DBM), sponge holding bone morphogenic protein (BMP), and allograft bone.

3. The posterior spine fixation apparatus of claim 1, wherein said tack is generally cylindrical and has a diameter in the range of 4-6 mm, and a length in the range from 10 mm to 30 mm adapted for insertion through an inferior medial facet of a spine vertebra and into a superior facet of a sacrum.

4. The posterior spine fixation apparatus of claim 1, wherein said tack is generally cylindrical and has a diameter in the range of 4-6 mm, and a length in the range of from 10 mm to 30 mm adapted for insertion through an inferior facet of a first spine vertebra and into a superior facet of a spine second vertebra.

5. The posterior spine fixation apparatus of claim 1, wherein said tack comprises a longitudinal passageway extending through the entire tack from the top end of the tack to the bottom end of the tack, for receiving a surgical guide-wire.

6. The posterior spine fixation apparatus of claim 1, wherein said apertures are selected from a group consisting of: rectangular apertures, circular apertures, oval apertures, pentagonal apertures, hexagonal apertures, and octagonal apertures.

7. The posterior spine fixation apparatus of claim 1, wherein the tack housing has multiple protrusions extending radially outward from said housing between or around said apertures.

8. The posterior spine fixation apparatus of claim 1, wherein the housing sidewall is selected from a group consisting of: stainless steel, tantalum, and polymer.

9. The posterior spine fixation apparatus of claim 1, wherein said tack has a cylindrical member upending from a top end of the tack, wherein said cylindrical member has a smaller diameter than the tack housing, the cylindrical member being for cooperation with a take holding tool.

10. The posterior spine fixation apparatus consisting only of two of said tacks, for insertion across a right facet joint of a lower spine and across a left facet joint of the lower spine, wherein said posterior spine fixation apparatus comprises no bars, no plates, no extensions, and no supports attached to, or extending from, the tacks.

11. A surgical method for spine fixation, the method comprising:
providing at least one elongated tack having a top end and a bottom end, the tack having a longitudinal passageway between said top and bottom end;
installing a guide-wire into a patient body and across a spine facet joint;
installing a sheath around the guide-wire and extending to the spine facet joint;
sliding a drill inside the sheath and along the guide-wire to said facet joint, wherein the drill has a longitudinal passageway the receives the guide-wire so that the drill is coaxial with the guide-wire;
drilling a hole across the facet joint and removing the drill from the sheath and from the guide-wire;
pushing said elongated tack through the sheath and into the hole, wherein the tack longitudinal passageway receives and slides along the guide-wire into the hole;
removing the guide-wire from the hole and patient.

12. The method as in claim 11, wherein said tack further comprises a hollow housing with an interior space and the hollow housing having a sidewall with apertures from an outermost surface of the sidewall to the interior space, the tack further comprising core material in said interior space, the core material comprising biologically-active material that encourages and receives bone growth through said apertures into the core material.

14. The method as in claim 12, wherein said biologically-active material is selected from the group consisting of: osteobiologic material, demineralized bone matrix (DBM), sponge holding bone morphogenic protein (BMP), and allograft bone.

15. The method as in claim 11, further comprising:
providing a plate for anterior fixation, and screwing the plate to anterior surfaces of two adjacent vertebrae, and wherein only said plate screwed to said anterior surfaces is used for fixation and fusing of anterior surfaces of said two adjacent vertebrae.

16. The method as in claim 11, wherein said tack has protrusions extending out radially from said sidewall of the housing.

17. The method as in claim 11, wherein said tack has no protrusions extending out radially from said sidewall of the housing.
